(19) United States
(12) Patent Application Publication
(10) Pub. No.: US 2003/0147928 A1
(43) Pub. Date: Aug. 7, 2003

(54) Antipruritic cosmetic and dermatological preparations

(75) Inventors: Dagmar Zelle, Bliedersdorf (DE); Alexander Filbry, Hamburg (DE); Rainer Kroepke, Schenefeld (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/172,241

(22) Filed: Jun. 14, 2002

(30) Foreign Application Priority Data
Nov. 27, 2001 (DE) 101 58 199.8

(51) Int. Cl. A61K 7/06; A61K 7/00

(52) U.S. Cl. 424/401; 424/74

(57) ABSTRACT

The present invention concerns antipruritic cosmetic and dermatological preparations that contain one or more O/W emulsifiers, optionally one or more co-emulsifiers, polidocanol and essential oil. Use of the preparation that is the subject of the present invention has been shown to have an immediate and long-lasting antipruritic effect.

### FIELD OF THE INVENTION

The present invention concerns antipruritic cosmetic and dermatological preparations that contain polidocanol and essential oil. When the preparation that is the subject of the present invention is applied, an immediate and long-lasting antipuritic effect is produced.

### BACKGROUND OF THE INVENTION

Itching (itchy skin, pruritus) is a subjectively irritating and unpleasant sensation relating to the skin or mucous membranes. It can be confined to a local area, or can also spread to affect the entire body. Itching can have a burning, stinging or tingling character, and almost always leads to rubbing or scratching. Such rubbing or scratching can then lead to scratch marks, open wounds, scabs, and skin infections. The itching is sensed via the skin's pain receptors, and this sensation is forwarded to the brain via the vegetative nervous system. The itching often has a complex cause, and can sometimes be the first and only symptom to appear in the case of certain skin diseases or general illnesses. However, in addition to dry skin, the failure to apply moisturisers or the effect of items of clothing, itching can also be caused by external factors and skin irritants, such as e.g. stings or mosquitoes or following contact with stinging nettles or jellyfish. Itching can be a reaction to chemical, mechanical or thermal irritants. It can be caused by external irritants such as e.g. by the effect of chemical substances, e.g. histamine (mosquito sting), apamine (bee sting), by allergic immune reaction, by pressure or rubbing or even by warmth or exposure to sunlight, weals, jellyfish stings and other skin reactions associated with itching. Another well-known and significant form of itching is the so-called senile pruritus, an unpleasant secondary condition which can affect people of advanced years.

When it comes to the treatment of itching, in addition to the diagnostic investigation of the cause, the principal focus tends to be on measures to reduce or eliminate the itching. This is often done without regard for the influence of the illness underlying the symptoms of itching.

The current medications or means used to alleviate the symptoms of itching are administered as tablets or in the form of powder, ointments, gels or emulsions. While tablets have a systemic effect that can burden the entire organism, in the case of gels, creams and emulsions a specific formulation is necessary in order to sustain the effectiveness of the active substance in the preparation.

There are numerous tried and tested treatments for itching. The majority of these involve the use of cooling gels and sticks, which—in addition to causing a cooling sensation through the evaporation of alcohol or water—also contain antihistamines. Isoprenalin is also used, although this can be a skin irritant and is consequently not recommended.

A well-known cream in this respect, for example, is Camillen 60 FUDES. The cream, which is based on Vaseline, calendula oil and menthol, is used to alleviate the symptoms of itching. Additional ingredients such as antibacterial substances, benzoic acid and triclosan provide protection primarily against fungal infection.

The painkilling gel Thesit®, which is distributed by the company Desitin, contains the ingredients polidocanol, mepivacain-hydrochloride and benzal conium chloride. The gel is intended for the treatment of skin injuries, burns and itching, whereby Mepivacain-hydrochloride acts as a local anaesthetic.

DE 19833177 describes the application of a plaster containing the active substances menthol and benzocain as a local anaesthetic to alleviate irritation in the case of insect bites or contact with stinging nettles. A disadvantage is that local anaesthetics can trigger contact allergies, and the addition of local anaesthetics means the product cannot later be distributed as a cosmetic.

### SUMMARY OF THE INVENTION

The purpose of the present invention is to make a preparation available that has a lasting antipuritic effect, which enriches the state-of-the art, and which offers an alternative to the previously known antipuritic preparations. A further purpose is to make an antipuritic preparation available which does not have the disadvantages of state-of-the-art preparations, and in particular which has an immediate and long-lasting antipuritic effect, without the need to administer an excessive quantity of antipuritic active substances. A further purpose is to make an antipuritic preparation available which has a skin-care effect and which is user-friendly in application. It must normalise the moisturising and moisture regulation of the skin, and must also provide the skin with protection against the rays of the sun. In particular, countries with high sun exposure also tend to have a higher risk of insect bites or contact with jellyfish.

The specified objectives are fulfilled by preparations in accordance with the principal claim. The object of the sub-claims are advantageous versions of the preparations which are the subject of the invention. Furthermore, the present invention also covers the application of such preparations.

It came as a surprise, and specialists were unable to anticipate that a preparation containing one or more O/W emulsifiers, possibly one or more co-emulsifiers, polidocanol and essential oil, would meet the set objectives.

In particular, the active ingredients polidocanol and menthol in non-ionic emulsion bases, in particular in emulsions based on eumulgin B-1, proved to be surprisingly effective.

### DETAILED DESCRIPTION OF THE PREFERRED EMBODIMENTS

The preparations which are the subject of the present invention lead to a significantly effective, long-lasting and subjectively perceptible reduction in skin itching.

Polidocanol is an abbreviation for polyethylenglykol(9)monododecylether, an adduct of 9 mol ethylene oxide on dodecyl alcohol; it corresponds to the formula

C₁₂H₂₅—(O—CH₂—CH₂)ₙ—OH

whereby n assumes an average value of 9 and the average mol weight is about 600 g/mol. Polidocanol is also known under the name nonaoxythylene monododecyl ether, PEG-9, lauryl ether, polyethylene glycol 450, lauryl ether polyoxyethylene (9). The trade names and marketing companies are inter alia Alfonic 1412-9.0 Ethoxylate (Condea Vista), Atlas G-4829 (Uniqema Americas), Calgene Nonionic L-9 (Calgene), Carsonon L-9 (Lonza Inc./Lonza Ltd.), Carsonon L-985 (Lonza Inc./Lonza Ltd.), Hetoxol L-9 (Heterene), Hetoxol LS-9 (Heterene), Jeecol LA-9 (Jeen), Marlipal 24/90 (Condea Chemie (Marl)), Nikkol BL-9EX (Nikko), Procol LA-9 (Protameen), Sympatens-AL/090 (Kolb), Unicol LA-9 (Universal Preserv-A-Chem).

The ability of the polidocanol to form an emulsion is not affected by the presence of electrolytes. Polidocanol an amphiphilic compound with high capillary activity. Aqueous solutions exhibit a high surface activity, and correspondingly good ability to spread on the skin. When applied in aqueous solutions, polidocanol exhibits strong local anaesthetic characteristics (K. Söhring & staff, Arch. Int. pharmacodyn. 87, 301 [1951]; K. J. Siems & K. Soehring, Arzneim.-Forschung./Drug Res. 2, 109 [1952]; K. Soehring & staff, Arch. Int. pharmacodyn. 91, 112 [1952]; H. S. Zipf & staff, Arzneim.-Forsch./Drug. Res. 7,162 [1957]).

Polidocanol is deployed in the preparation in a proportion of 0.01 to 20 w/w %, in particular between 1 and 10 w/w %.

Amongst essential oils, concentrates derived from plants are known which are deployed as natural raw materials predominantly in the perfume and food industries, and which consist more or less of volatile compounds, such as for example genuine essential oils, citrus oils, absolute essences, resinoids.

The term is also often applied to other volatile substances that the plants contain. Strictly speaking, however, essential oils are mixtures of volatile components that are produced by the steam distillation of plant raw materials.

Genuine essential oils consist exclusively of volatile components with boiling points which tend to lie between 150 and 300 C. This means that unlike, for example, greasy oils, they do not leave any permanent transparent grease stains when applied to filter paper. Essential oils contain mostly hydrocarbons or monofunctional compounds such as aldehydes, alcohols, esters, ethers and ketones.

Parent compounds include monoterpenes and sesquiterpenes, phenylpropane derivatives and longer-chain aliphatic compounds.

Some essential oils have a dominant ingredient (for example eugenol, which makes up over 85% of clove oil), while others have extremely complex compositions. The organoleptic characteristics are often caused not by the principal components, but instead by secondary or trace components, such as for example the 1,3,5-undecatrienes and pyrazines in galbanum oil. In the case of many commercially important essential oils, the number of identified components go into the hundreds. Many ingredients are chiral, whereby it is often the case that one enantiomer dominates or preponderates or is exclusively present, such as for example (−)-menthol in peppermint oil or (−)-linalylacetate in lavender oil.

Menthol has been shown to be a particularly favoured essential oil. Menthol is a component of the well-known Japanese peppermint oil (CAS: 20747-49-3). The most important isomer is (−)-menthol. When rubbed onto the skin, for example to treat migraine or similar complaints, menthol generates a pleasant cooling sensation. This is caused by surface anaesthesia and the stimulation of cold-sensitive nerves. It has been demonstrated that the temperature of skin areas treated in this way is normal or raised.

Other favoured essential oils which could be listed include Oleum Eucalypti, Oleum Menthae piperitae, Oleum camphoratum, Oleum Rosmarini, Oleum Thymi, Oleum Pini sibricum and Oleum Pini silverstris as well as the terpenes 1.8-Cineol and Levomethanol as well as Oleum Abietis albae, Oleum Anisi, Oleum Aurantii Floris, Oleum Bergamottae, Oleum Calendulae infusum, Oleum Caryophylli, Oleum Chamomillae, Oleum Cinnamomi ceylanici, Oleum Citri, Oleum Citronellae, Oleum Cupressi, Oleum Cymbopogonis, Oleum Jecoris, Oleum Lavendulae, Oleum Macidis, Oleum Majoranae, Oleum Melaleucae viridiflorae, Oleum Melissae, Oleum Menthae arvensis, Oleum Millefolium, Oleum Myrrhae, Oleum Myrte, Oleum Pini sibricum, Oleum Pinisilvestris, Oleum Salviae, Oleum Santali, Oleum Terebinthinae rectificat., Oleum Valerianae and Oleum Zingiberis.

The essential oils are deployed in the preparation either individually or in combination with others, comprising a total of between 0.001 and 10 w/w %, in particular between 0.01 and 1 w/w %.

The most common form in which cosmetic or dermatological preparations appear are fine-dispersal multi-phase systems which have one or more fat or oil phases in addition to one or more water phases. Of these systems, actual emulsions are the most widespread.

In simple emulsions, one phase contains finely-dispersed droplets surrounded by an emulsifier coating, while the second phase (water droplets in W/O or lipid vesicles in O/W emulsions). The droplet diameter of normal emulsions lies in the range of approx. 1 μm to approx. 50 μm. Such “macro-emulsions” are, without further colouring additives, milky-white in colour and opaque. By contrast, finer “macro-emulsions” with droplet diameters of approx. 10⁻¹ μm to ca. 1 μm are, again in the absence of colouring additives, blue-white in colour and non-transparent.

Micellar and molecular solutions with particle diameters smaller than approx. 10⁻² μm, on the other hand, appear clear and transparent.

The droplet diameter of transparent or translucent micro-emulsions, by contrast, lies in the range of approx. 10⁻² μm to approx. 10⁻¹ μm. Such micro-emulsions tend to have low viscosity. The viscosity of many micro-emulsions of the O/W type is comparable with that of water.

The advantage of micro-emulsions is that in the dispersal phase the active substances can be dispersed more finely than in the dispersal phase of “macro-emulsions.” A further advantage is that their low viscosity means they can be applied in the form of a spray. A disadvantage of current state-of-the-technology micro-emulsions is that they always need to contain a high proportion of one or more emulsifiers, as the low droplet diameter results in a high boundary surface forming between the phases, which generally has to be stabilised using emulsifiers.

With respect to the invention, the favoured O/W emulsifiers include Eumulgin B-1 from the company Cognis, an emulsifier base corresponding to the names polyethylenglycol(12)cetearylether, PEG-12 cetyl/stearyl ether, polyethylene glycol 600 cetyl/stearyl ether, polyoxyethylene (12) cetyl/stearyl ether. This compound is also known by the name of CETEARETH-12, and is marketed under the following names by the following companies: Atlas G-4822 (Uniqema Americas), Jeecol CS-12 (Jeen), Procol CS-12 (Protameen), Sabowax CS 11 (Sabo), Sympatens-ACS/120 (Kolb), Unimul-B-1 (Universal Preserv-A-Chem), Volpo CS12 (Croda Oleochemicals).

Another advantageous substance is Myrj 52S from the company Uniqema. This is an emulsifier corresponding to the names Macrogol Stearate 2000, polyethylene glycol 2000, monostearate polyoxyethylene (40), monostearate polyoxyl 40, stearate stearethate 40. This compound is also known by the name PEG-40 STEARATE, and is marketed under the following names by the following companies: AEC PEG-40 Stearate (A & E Connock) Calgene POE (40) MS (Calgene) Crodet S40 (Croda Oleochemicals) Emerest 2715 (Henkel) Emerest 2715 (Henkel/COSPHA) Hetoxamate SA-40 (Heterene) Jeemate 2000-DPS (Jeen) Lanoxide-52 (Lanaetex) LIPOPEG-39-S (Lipo) Myrj 52 (Uniqema Americas) Myrj 52S (Uniqema Americas) Nikkol MYS-40 (Nikko) Pegosperse 1750 MS (Lonza Inc./Lonza Ltd.) Protamate 1540 DPS (Protameen) Protamate 2000 DPS (Protameen) Ritox 52 (RITA) ROL 52 (Fabriquimica) Sabowax SE 40 (Sabo) Simulsol M 52 (SEPPIC) Sipoic MS-40 (Specialty Industrial) Sympatens-BS/400 (Kolb) Tego Acid S 40 P (Goldschmidt) Unipeg-S-40 (Universal Preserv-A-Chem).

The substance Cutina GMS (from the company Cognis) has proven to be a very effective co-emulsifier. This co-emulsifier corresponds to the name 2,3-dihydroxypropyl octadecanoate, glycerine 1-stearate, glyceryl monostearate, glycerol 1-stearate, Jeechem HMS monostearine, octadecanoic acid, 2,3-dihydroxypropyl ester octadecanoic acid, monoester with 1,2,3-propanetriol stearic acid 1-monoglyceride. This compound is also known by the name GLYCERYL STEARATE and is also marketed under the names AEC Glyceryl Stearate (A & E Connock) Aldo HMS (Lonza Inc./Lonza Ltd.) Aldo MS (Lonza Inc./Lonza Ltd.) Aldo MSLG (Lonza Inc./Lonza Ltd.) Alkamuls GMS (Rhodia Inc.) Arlacel 129 (Uniqema Americas) Arlacel 161 (Uniqema Americas) Arlacel 169 (Uniqema Americas) Calgene GMS (Calgene) Capmul GMS (Abitec) Ceral Mex (Fabriquimica) Ceral MN (Fabriquimica) Ceral MNT (Fabriquimica) Cerasynt GMS (ISP Van Dyk) Cerasynt SD (ISP Van Dyk) Cithrol GMS N/E (Croda Oleochemicals) CPH-53-N (Hall) CPH-144-N (Hall) Cremophor GS 11 (BASF) Cutina GMS (Cognis) Emerest 2400 (Henkel) Emerest 2400 (Henkel/) ESTOL 1474 (Uniqema (Netherlands)) ESTOL 3740 (Uniqema (Netherlands)) Geleol (Gattefosse s.a.) Imwitor 191 (Condea Chemie (Witten)) Imwitor 900 (Condea Chemie (Witten)) Jeechem GMS-450 (Jeen) Jeechem HMS (Jeen) Kemester 5500 (Witco) Kemester 6000 (Witco) Kessco GMS (Akzo Nobel Surface Chemistry) KESSCO GMS (Stepan) KESSCO GMS 63F (Stepan) KESSCO GMS PURE (Stepan) Lanesta 24 (Lanaetex).

Further O/W emulsifiers or co-emulsifiers include polypropoxylised O/w emulsifiers

whereby the polyethooxylised or polypropoxylised O/W co-emulsifiers and/or emulsifiers are selected from the group

the fatty alcohol ethoxylates of the general formula R—O—(—CH₂—CH₂—O—)ₙ—H, whereby R represents a branched or unbranched alkyl, aryl or alkenyl residue and n represents a number between 10 and 50

the ethoxylised wool wax alcohols,

the polyethylenglycol ethers of the general formula R—O—(—CH₂—CH₂—O—)ₙ—R′, whereby R and R′ represent independently branched or unbranched alkyl or alkenyl residues and n represents a number between 10 and 80

the fatty acid ethoxylates of the general formula R—COO—(—CH₂—CH₂—O—)ₙ—H, whereby R represents a branched or unbranched alkyl or alkenyl residue and n represents a number between 10 and 40,

the etherised fatty acid ethoxylates of the general formula R—COO—(—CH₂—CH₂—O—)ₙ—R′, whereby R and R′ represent independently branched or unbranched alkyl or alkenyl residues and n represents a number between 10 and 80,

the esterised fatty acid ethoxylates of the general formula R—COO—(—CH₂—CH₂—O—)ₙ—C(O)—R′, whereby R and R′ represent independently branched or unbranched alkyl or alkenyl residues and n represents a number between 10 and 80,

the polyethylene glycolglycerine fatty acid esters of saturated and/or unsaturated, branched and/or unbranched fatty acids and a degree of ethoxylation between 3 and 50,

the ethoxylised sorbitane esters with a degree of ethoxylation between 3 and 100

the cholesterine ethoxylates with a degree of ethoxylation between 3 and 50,

the ethoxylised triglycerides with a degree of ethoxylation between 3 and 150,

the alkylether carboxylic acids of the general formula R—O—(—CH₂—CH₂—O—)ₙ—CH₂—COOH or their cosmetic or pharmaceutically acceptable salts, whereby R represents branched or unbranched alkyl or alkenyl residues with 5-30 C atoms and n represents a number between 5 and 30,

the polyoxyethylene sorbitol fatty acid esters, based on branched or unbranched alkanoic or alkenoic acids and a degree of ethoxylation of 5 to 100, for example of the sorbeth type,

the alkylether sulphates or the acids which are based on these sulphates of the general formula R—O—(—CH₂—CH₂—O—)ₙ—SO₃—H with acceptable cosmetic or pharmaceutical, whereby R represents branched or unbranched alkyl or alkenyl residues with 5-30 C atoms and n represents a number between 1 and 50.

the fatty alcohol propoxylates of the general formula R—O—(—CH₂—CH(CH₃)—O—)ₙ—H, whereby R represents branched or unbranched alkyl or alkenyl residues and n represents a number between 10 and 80,

the polypropylene glycolethers of the general formula R—O—(—CH₂—CH(CH₃)—O—)ₙ—R′, whereby R and R′ represent independently branched or unbranched alkyl or alkenyl residues and n represents a number between 10 and 80

the propoxylised wool wax alcohols,

the etherised fatty acid propoxylates of the general formula R—COO—(—CH₂—CH(CH₃)—O—)ₙ—R′, whereby R and R′ represent independently branched or unbranched alkyl or alkenyl residues and n represents a number between 10 and 80,

the esterised fatty acid propoxylates of the general formula R—COO—(—CH₂—CH(CH₃)—O—)ₙ—C(O)—R′, whereby R and R′ represent independently branched or unbranched alkyl or alkenyl residues and n represents a number between 10 and 80,

the fatty acid propoxylates of the general formula R—COO—(—CH₂—CH(CH₃)—O—)ₙ—H, whereby R represents a branched or unbranched alkyl or alkenyl residue and n represents a number between 10 and 80,

the polypropylene glycolglycerine fatty acid esters of saturated and/or unsaturated, branched and/or unbranched fatty acids and a degree of propoxylisation of between 3 and 80

the propoxylised sorbitane esters with a degree of propoxylisation of between 3 and 100

the cholesterine propoxylates with a degree of propoxylisation of between 3 and 100

the propoxylised triglycerides with a degree of propoxylisation of between 3 and 100

the alkylether carboxylic acids of the general formula R—O—(—CH₂—CH(CH₃)O—)ₙ—CH₂—COOH or their cosmetic or pharmaceutically acceptable salts, whereby R represents a branched or unbranched alkyl or alkenyl residue and n represents a number between 3 and 50,

the alkylether sulphates or the acids on which these sulphates are based of the general formula R—O—(—CH₂—CH(CH₃)—O—)ₙ—SO₃—H with cosmetic or pharmaceutically acceptable cations, whereby R represents a branched or unbranched alkyl or alkenyl residue with 5-30 C atoms and n represents a number between 1 and 50,

the fatty alcohol ethoxylates/propoxylates of the general formula R—O—Xₙ—Yₘ—H, whereby R represents a branched or unbranched alkyl or alkenyl residue, whereby X and Y are not identical and each represent either an oxyethylene group or an oxypropylene group, while n and m represent independent figures of between 5 and 50,

the polypropylene glycolethers of the general formula R—O—Xₙ—Yₘ—R′, whereby R and R′ represent independently branched or unbranched alkyl or alkenyl residues, whereby X and Y are not identical and each represent either an oxyethylene group or an oxypropylene group, while n and m represent independent figures of between 5 and 100,

the etherised fatty acid propoxylates of the general formula R—COO—Xₙ—Yₘ—R′, whereby R and R′ represent independently branched or unbranched alkyl or alkenyl residues, whereby X and Y are not identical and each represent either an oxyethylene group or an oxypropylene group, while n and m represent independent figures of between 5 and 100,

the fatty acid ethoxylates/propoxylates of the general formula R—COO—Xₙ—Yₘ—H, whereby R represents a branched or unbranched alkyl or alkenyl residue, whereby X and Y are not identical and each represent either an oxyethylene group or an oxypropylene group, while n and m represent independent figures of between 5 and 50.

It is advantageous to select the fatty alcohol ethoxylates from the group of the ethoxylised stearyl alcohols, cetyl alcohols, cetylstearyl alcohols (cetearyl alcohols). The following are particularly favoured:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glyol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleylether (oleth-12), polyethylene glycol(13) oleylether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolaurylether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), polyethylene glycol(20) cetylstearyl ether (ceteareth-20).

It is furthermore advantageous to select the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

It can be advantageous to use sodium laureth-11-carboxylate as ethoxylised alkyl ether carboxylic acid or its salt.

It can be advantageous to use sodium laureth 1-4 sulphate as alkyl ether sulphate. Polyethylene glycol(30) cholesteryl ether as ethoxylised cholesterine derivative can also be used to advantage. Polyethylene glycol(25) sojasterol has also proven successful. Polyethylene glycol(60) evening primrose glycerides can be used as ethoxylised triglycerides.

It is also advantageous to select the polyethylene glycol glycerine fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, polyethylene glycol(18) glycery loleate/cocoate.

It is also favourable to select the sorbitane esters from the group polyethylene glycol(20) sorbitane monolaurate, polyethylene glycol(20) sorbitane monostearate, polyethylene glycol(20) sorbitane monoisostearate, polyethylene glycol(20) sorbitan monopalmitate, and polyethylene glycol(20) sorbitane monooleate.

In the case of the preparations that are the subject of the invention, an unexpected synergy develops between the effects of the active substances, brought about by the combination of the active substances polidocanol and essential oils, in particular menthol, with the selected O/W emulsifiers. The selected O/W emulsifiers create a gel network in the coherent aqueous phase in a form which favours the release of the active substances polidocanol and essential oil in such a way that the antipruritic effect can develop in an immediate yet sustained manner.

The results of the clinical assessment of the effectiveness of the preparation that is the subject of the present invention are extremely positive, and confirm the advantageous characteristics of the preparations. Within the framework of a clinically controlled study, the preparations which are the subject of the present invention were tested with 1 w/w % menthol and 2 w/w % polidocanol, 0.5 w/w % menthol and 2 w/w % polidocanol and a placebo on several individuals of between 18 and 60 years of age.

The result:

Immediate reduction in itching following the application of the preparation

Intensification of the reduction in itching within 30 minutes following application, up to almost entire elimination of the irritation

Following one week of treatment, also perceptible reduction in itching

No difference between the various applied preparations which are the subject of the present invention

In addition to water, further ingredients of the preparations that are the subject of the present invention are standard substances used in cosmetics as described in greater detail below.

For the oil phase of the preparation that is the subject of the present invention, oils with different polarity, molecular weight and structure can be deployed. The oil component or the entirety of the oil components of the preparation which is the subject of the present invention are chosen preferably from the group of esters of saturated and/or unsaturated, branched and/or unbranched alkane carboxylic acids or hydroxyalkane carboxylic acids with a chain length of 1 to 44 C atoms and saturated and/or unsaturated, branched and/or unbranched alcohols with a chain length of 1 to 44 C atoms from the group of esters of aromatic carboxylic acids and saturated and/or unsaturated, branched and/or unbranched alcohols with a chain length of 1 to 30 C atoms, insofar as the oil component or the entirety of the oil components form a liquid at room temperature. Such ester oils can likewise be selected beneficially from the group isopropylmyristate, isopropylpalmitate, isopropylstearate, isopropyloleate, n-butylstearate, n-hexyllaurate, n-decyloleate, isooctylstearate, isononylstearate, isononylisononanoate, 2-ethylhexylpalmitate, 2-ethylhexyllaurate, 2-hexyldecylstearate, 2-octyldodecylpalmitate, oleyloleate, oleylerucate, erucyloleate, erucylerucate as well as synthetic, semi-synthetic and natural mixtures of such esters, e.g. jojoba oil.

The oil phase can be selected beneficially from the group of branched and unbranched hydrocarbons, the silicon oils, lanolins, the adipic acid esters, the butylene glycol diesters, the dialkyl ethers or carbonates, the group of the saturated or unsaturated, branched alcohols, as well as the fatty acid triglycerides, specifically the triglycerine esters of saturated and/or unsaturated, branched and/or unbranched alkane carboxylic acids with a chain length of 8 to 24, in particular 12-18 C atoms. The fatty acid triglycerides can, for example, be selected beneficially from the group of the synthetic, semisynthetic and natural oils, e.g. olive oil, sunflower oil, soya oil, peanut oil, rape seed oil, almond oil, palm oil, coconut oil, palm kernel oil and similar.

Furthermore, it can be advantageous for the oil phase to contain cyclical or linear silicone oils, or to consist entirely of such oils, whereby, however, in addition to the silicone oil or the silicone oils, it is preferable to use an additional component of other oil phase components.

It is advantageous to use cyclomethicone (octamethylcyclotetrasiloxane) as the silicone oil within the context of the present invention. However, other silicone oils can also be used to advantage within the context of the present invention, for example hexamethylcyclotrisiloxane, polydimethylsiloxane, poly(methylphenylsiloxane).

Very advantageous are for example dicapylylcarbonate, dicaprylylether, paraffin oil, coco caprylate/caprate, caprylic/capric triglyceride, cyclomethicone, dimethicone, octyldodecanol as well as natural oils such as e.g. soya oil, macadamia oil, evening primrose oil. Furthermore, other very advantageous oil blends can also be deployed, such as for example paraffin oil/dicaprylyether, paraffin oil/coco caprylate caprate.

Other advantageous oil components include e.g. butyloctylsalicylate (for example available under the trade name Hallbrite BHB from the company CP Hall), hexadecylbenzoate and butyloctylbenzoate and mixtures thereof (Hallstar AB) and/or diethylhexylnaphthalate (Hallbrite TQ).

The oil components can be used advantageously in a content of 1 to 50 w/w % with respect to the overall preparation, whereby approx. 1 to 40 w/w % is favoured.

Through the use of waxes (ester waxes, triglyceride waxes, ethoxlised waxes etc.) in the oil phase or as the oil phase, the preparation can be improved still further. It is preferable to select the wax components or the entirety of the wax components from the group of esters of saturated and/or unsaturated, branched and/or unbranched alkane carboxylic acids or hydroxy carboxylic acids with chain lengths of 1 to 80 C atoms and saturated and/or unsaturated, branched and/or unbranched alcohols with chain lengths of 1 to 80 C atoms, from the group of esters of aromatic carboxylic acids and saturated and/or unsaturated, branched and/or unbranched alcohols with chain lengths of 1 to 80 C atoms, insofar as the wax components or the entirety of the wax components form solid bodies at room temperature, the natural waxes, the diesters of polyols and C10-C80 fatty acids, the ethoxylised waxes, the triglyceride waxes, the C16-C60 fatty acids and/or C16-C80 fatty alcohols, the mineral oil waxes.

It is particularly advantageous to select the wax components from the group of the C₁₆₋₃₆-alkylstearates, the C₁₀₋₄₀-alkylstearates, the C₂₀₋₄₀-alkylisostearates, the C₂₀₋₄₀-dialkyldimerates, the C₁₈₋₃₈-alkylhydroxystearoylstearates, the C20-40 dialkyldimerates, the C₂₀₄₀-alkylerucates, and furthermore C₃₀₋₅₀-alkyl bees wax, cetylpalmitate, methylpalmitate, cetearylbehenate, and octacosanyl stearate. Silicone waxes, such as for example, stearyltrimethylsilane/stearylalcohol may also be advantageous.

Also advantageous within the meaning of the present invention are ester waxes, comprising esters of

1. saturated and/or unsaturated, branched and/or unbranched mono and/or dicarboxylic acid with 10 to 50 carbon atoms, preferably 15-45 carbon atoms and

2. glycerine.

In this respect, mono, di and triglycerides can be advantageous.

The following listed glycerides are particularly advantageous:

| Glyceride | Trade name | Available from |
|---|---|---|
| C₁₆₋₁₈-triglyceride | Cremeol HF-52-SPC | Aarhus Oliefabrik |
| Glycerylhydroxystearate | Naturchem GMHS | Rahn |
| Hydrated coco-glycerides | Softisan 100 | Hüls AG |
| Caprylic acid/capric acid/isostearine acid/ | Softisan 649 | Dynamit Nobel |
| adipine acid triglyceride | | |
| C₁₈₋₃₆ triglyceride | Syncrowax HGLC | Croda GmbH |
| Glyceryltribehenate | Syncrowax HRC | Croda GmbH |
| Glyceryl-tri-(12-hydroxystearate) | Thixcin R | Rheox/NRC |
| Hydrated castor oil | Cutina HR | Cognis AG |
| C₁₆₋₂₄-triglyceride | Cremeol HF-62-SPC | Aarhus Oliefabrik |

It is particularly preferred to select the wax components from the group of triglyceride waxes, such as C18-38 triglyceride or tribehenine. Furthermore, it has been shown that ethoxylised waxes such as for example PEG-8 bees wax, PEG 6 sorbitane bees wax, PEG-2 hydrogenated castor oil, PEG-12 carnauba wax are advantageous.

In addition, the preparations that are the subject of the present invention can contain colouring agents and/or pigments. The colouring agents and pigments can be selected from the corresponding positive list of the German Cosmetics Code [Kosmetikverordnung] or from the EU list of cosmetic colouring agents. In the majority of cases, these are identical to the colouring agents approved for use in food. Pigments can be of organic and inorganic origin, such as for example organic pigments of the azo type, indigoides, triphenylmethane-type, anthrachinones, and xanthine colouring agents which are known as D&C and FD&C blues, browns, greens, oranges, reds, yellows. Inorganic pigments consist of insoluble salts of certified colouring agents, known as lakes iron oxides. For example, barium lakes, calcium lakes, aluminium lakes, titanium dioxides, mica and iron oxides can be used. In the case of aluminium sales, for example, red 3 aluminium lake, red 21 aluminium lake, red 27 aluminium lake, red 28 aluminium lake, red 33 aluminium lake, yellow 5 aluminium lake, yellow 6 aluminium lake, yellow 10 aluminium lake, orange 5 aluminium lake, blue 1 aluminium lake and combinations thereof can be deployed.

In the case of iron oxides or oxidhydrates, for example, cosmetic yellow oxide C22-8073 (Sunchemical), cosmetic oxide MC 33-120 (Sunchemical), cosmetic brown oxide C33-115 (Nordmann& Rassmann), cosmetic russet oxide C33-8075 (Sunchemical) are known and may be advantageous. As alumosilicate, ultramarine blue (Les colorants Wacker) can be deployed.

Pearl lustre pigments can also be used in the emulsions that are the subject of the present invention. These are available, for example, from the companies Costenoble (Cloisonne Type, Flamenco Type, Low Lustre Type), Merck (Colorona Types, Microna Type, Timiron Type, Colorona, Ronasphere), Les Colornats Wacker (Covapure, Vert oxyde de Chrome), Cadre (Colorona, Sicopearl), BASF (Sicopearl, Sicovit), Rona (Colorona). For example, Timiron Silk Gold and Colorona Red Gold have been shown to be particularly advantageous pearl lustre pigments.

Other advantageous colour pigments include titanium dioxide, mica, iron oxide (e.g. Fe₂O₃, Fe₃O₄, FeO(OH)) and/or tin oxide. Advantageous colouring agents include for example carmine, Berlin blue, chrome oxide green, ultramarine blue and/or manganese violet. It is in particular advantageous to select the colouring agents and/or colour pigments from the following list. (The substances are ordered in accordance with their colour index number.)

| Colour Index | | CAS No. or empirical |
|---|---|---|
| Number | German Name | formula |
| 10316 | C-ext. Gelb 1 | 846-70-8 |
| 12075 | C-Orange | 3468-63-1 |
| 14700 | C-Rot 57 | 4548-53-2 |
| 15510 | C-ext. Orange 8 | 633-96-5 |
| 15585 | C-Rot 55 | 2092-56-0 |
| 15585:1 | C-Rot 55 | 5160-02-1 |
| 15800:1 | C-ext. Rot 57 | 6371-76-2 |
| 15850 | Lithol Rubin 8 | 5858-81-1 |
| 15850:1 | C-Rot 12 | 5281-04-9 |
| 15880:1 | C-ext. Rot 61 | 6417-83-0 |
| 15980 | C-Orange 9 | C₁₆H₁₀N₂O₇S₂.2Na |
| 15985 | C-Orange 10 | 2783-94-0 |
| 16035 | C-Rot 60 | 29956-17-6 |
| 17200 | C-Rot 58 | C₁₆H₁₃N₃O₇S₂.2Na |
| 19140 | C-Gelb 10 | 1934-21-0 |
| 20170 | C-ext. Braun 4 | 1320-07-6 |
| | | 6371-84-2 |
| 26100 | C-ext. Rot 56 | 85-86-9 |
| 42053 | C-Grün 12 | C₃₇H₃₆N₂O₁₀S₃.2Na |
| 42090 | C-Blau 21 | 2650-18-2 |
| 42090 | C-Blau 21 | 2650-18-2 |
| | (Ammonium Salz) | 6371-85-3 |
| | | 37307-56-5 |
| 45170 | C-Rot 59 | 81-88-9 |
| 45170:1 | (Rhodamin B-stearat) | C₂₈H₃₁N₂O₃.C₁₈H₃₅O₂ |
| 45370:1 | C-Rot 27 | C₂₀H₁₀Br₂O₅ |
| 45380 | C-Rot 30 | 17372-87-1 |
| 45380:2 | Tetrabromfluoreszein | 15086-94-9 |
| 45410 | C-Rot 34 | 18472-87-2 |
| 45410:1 | Tetrabromtetrachlorfluoreszein | 13473-26-2 |
| 45425 | C-Rot 35 | C₂₀H₁₀I₂O₅.2Na |
| 45425:1 | Fluoreszein-Gemisch | 518-40-7 |
| | | 38577-97-8 |
| 47000 | C-ext. Gelb 23 | 8003-22-3 |
| 47005 | C-Gelb 11 | 8004-92-0 |
| 59040 | C-ext. Gelb 24 | 6358-69-6 |
| 60725 | C-ext. Violett 18 | 81-48-1 |
| 61565 | C-Grün 10 | 128-80-3 |
| 61570 | C-Grün 11 | 4403-90-1 |
| 73360 | C-Rot 28 | 2379-74-0 |
| 75120 | C-Orange 12 | 8015-67-6 |
| 75130 | C-Orange 11 | 7235-40-7 |
| 75170 | Guanin | 68-94-0 |
| | | 73-40-50 |
| 75470 | C-Rot 50 | C₂₂H₂₀O₁₃ |
| 75480 | Henna | C₁₀H₆O₃ (Lawson) |
| 75810 | C-Grün 8 | 11006-34-1 |
| 75810 | C-Grün 7 | 479-61-8 |
| | | 519-62-0 |
| 77000 | C-Pigment 1 | Al |
| 77007 | C-Blau 16 | 57455-37-5 |
| 77019 | C-Weiβ 11 | 12001-26-2 |
| 77288 | C-Grün 9 | 1308-38-9 |
| 77289 | C-Grün 14 | 12001-99-9 |
| 77400 | Bronze | 7440-50-8 |
| 77491 | C-Rot 45 | 1309-37-1 |
| 77492 | C-Braun 3 | Fe₂O₃FeO(OH) |
| | (C-Gelb 8) | |
| 77499 | C-Schwarz 5 | Fe₃O₄ |
| 77510/20 | C-Blau 17 | C₆FeN₆.4/3 Fe |
| 77742 | C-Violett 11 | 10101-66-3 |
| 77820 | C-Pigment 2 | 7440-22-4 |
| 77891 | C-Weiβ 7 | 13463-67-7 (TiO₂) |
| 77947 | C-Weiβ 8 | 1314-13-2 |

The colouring agents and pigments can also be deployed individually, blended or used in layers, thereby creating different colour effects through general layers of varying thickness.

Of course, the list of the aforementioned colouring agents and colour pigments that can be used in the emulsions that are the subject of the present invention is not restricted.

Preparations that are the subject of the present invention can also contain powdered substances. For example, powdered substances can be used composed of bismuth oxichloride, titaniumised mica, silicium dioxide (fumed silica), spherical silicium dioxide pearls, polymethylmethacyrlate pearls, micronised Teflon, boron nitride, acrylate polymers, aluminium silicate, aluminium starch octenylsuccinate, bentonite, calcium silicate, cellulose, chalk, maize starch, glyceryl starch, hectorite, hydrogenated silica, kaolin, magnesium hydroxides, magnesium oxide, magnesium silicates, magnesium trisilicate, maltodextrine, montmorillonite, microcristalline cellulose, rice starch, silica, talcum, mica, titanium dioxide, tin laurates, tin myristate, tin neodecanoate, tin rosinate, tin stearate, polyethylene, aluminium oxide, attapulgite, calcium carbonate, calcium silicate, dextrane, kaolin, Nylon, silica silylate, silk powder, serecite, tin oxide, titanium hydroxide, trimagnesium phosphate, walnut shell powder, or any desired combination thereof.

The aforementioned standard cosmetic additives make it possible to apply the preparation to the skin for long periods, without a perceptible decline in the efficacy of the preparation.

Furthermore, additional advantageous active substances can be added to the preparations. For example, ribwort tincture has been shown to boost the antipruritic efficacy of the preparation relative to preparations that do not contain ribwort tincture.

In addition, skin-care active substances can be added to the preparations that are not restricted to fat-soluble active substances. These can be chosen from the group of water-soluble active substances, for example, vitamins and similar substances. This can produce a skin-care effect, even when the preparation is applied for a lengthy period.

A remarkable characteristic of the preparations which are the subject of the present invention is that these act as excellent vehicles for transporting cosmetics or active dermatological substances to the skin, whereby the favoured active substances in this respect are antioxidants which can protect the skin from the effect of oxidants.

For this reason, it is advantageous for the preparations to contain one or several antioxidants. All antioxidants that are suitable or usual for cosmetic and/or dermatological applications can be deployed as favourable, but nevertheless optional antioxidant additives in the preparations.

In this respect, particularly advantageous antioxidants are chosen from the group consisting of amino acids (e.g. glycine, histidine, tyrosine, tryptophane) and derivatives, imidazoles (e.g. urocanic acid) and derivatives, peptides such as D,L-carnosine, D-carnosine, L-carnosine and derivatives (e.g. anserine), carotinoides, carotines (e.g. α-carotine, β-carotine, lycopine) and derivatives, lipoic acid and derivatives (e.g. dihydrolipoic acid), aurothioglucose, propylthiouracil and other thioles (e.g. thioglycerine, thiosorbitol, thioglycollic acid, thioredoxine, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) as well as their salts, dilaurylthiodipropionate, distearylthiodipropionate, thiodipropionic acid and derivatives (esters, ethers, peptides, lipids, nucleotides, nucleosides and salts) as well as sulphoximine compounds (e.g. buthioninsulphoximine, homocysteinsulphoximine, buthioninsulphones, pentat, hexa, heptahioninsulphoximine) in very low tolerable dosages (e.g. pmol to μmol/kg), as well as (metal) chelatores (e.g. α-hydroxy fatty acids, palmitic acid, phytinic acid, lactoferrin), α-hydroxy acids (e.g. citric acid, lactic acid, malic acid), humic acid, bile acid, gall extracts, bilirubin, biliverdin, EDTA, EGTA and derivatives, unsaturated fatty acids and their derivatives (e.g. gamma linolenic acid, linolic acid, oleic acid), folic acid and derivatives, ubichinon and ubichinol and derivatives, vitamin C and derivatives (e.g. ascorbylpalmitate, Mg-ascorbylphosphate, ascorbylacetate), isoascorbic acid and derivatives, tocopheroles and derivatives (e.g. vitamin E-acetate), vitamin A and derivatives (vitamin A-palmitate) as well as coniferylbenzoate of benzoe resin, rutinic acid and derivatives, ferulic acid and derivatives, butylhydroxytoluol, butylhydroxyanisol, nordihydroguajak resin acid, nordihydroguajaretic acid, trihydroxybutyrophenone, uric acid and derivatives, mannoses and their derivatives, tin and derivatives (e.g. ZnO, ZnSO₄), selenium and derivatives (e.g. selenium methionine), stilbenes and derivatives (e.g. stilbenoxide, trans-stilbenoxide) and the derivatives of the aforementioned active substances which are suitable within the framework of the present invention (salts, esters, ethers, sugars, nucleotides, nucleosides, peptides and lipids).

Insofar as vitamin E and/or its derivatives represent the antioxidant(s), then it is advantageous to select their respective concentration from the range 0.001-10 w/w %, in relation to the total weight of the formulation.

Insofar as vitamin A, or vitamin-A derivatives, or carotines or their derivatives represent the antioxidant(s), then it is advantageous to select their respective concentration from the range 0.001-10 w/w %, in relation to the total weight of the formulation.

Within the framework of the invention, active substances can also be selected very advantageously from the group of the lipophile active substances, in particular from the following group:

Acetylsalicylic acid, atropine, azulene, hydrocortisone and derivatives, e.g. hydrocortisone-17-valerate, vitamins, e.g. ascorbic acid and derivatives, vitamins in the B and D group, the particularly favourable vitamin B₁, vitamin B₁₂, vitamin D₁, as well as bisabolol, unsaturated fatty acids, specifically the essential fatty acids (often called vitamin F), in particular y-linolenic acid, oleic acid, eicosapentaenic acid, docosahexaenic acid and derivatives, chloramphenicol, caffeine, prostaglandins, thymol, camphor, extracts or other products of plant or animal origin, e.g. evening primrose oil, borage oil or red currant seed oil, fish oils, liver oil, or ceramides and ceramide-like compounds, etc.

It is also advantageous to select the active substances from the group of fat-restoring substances, for example Purcellinöl®, Eucerit® and Neocerit®.

It is particularly advantageous to select the active substance or substances from the group of the NO synthesis inhibitors, in particular if the preparations which are the subject of the present invention are to be used to treat and to prevent the symptoms of intrinsic and/or extrinsic skin ageing, as well as to treat and provide protection against the harmful effects of ultraviolet radiation on the skin. Favoured NO synthesis inhibitor is nitroarginine.

It is also advantageous to select active substances from the group comprising catechins and bile acid esters of catechins and aqueous or organic extracts from plants or parts of plants which contain catechins or bile acid esters of catechins, such as for example the leaves of the plant family Theaceae, in particular the species Camellia sinensis (green tea). Particularly advantageous are their typical ingredients (such as e.g. polyphenols or catechins, caffeine, vitamins, sugar, minerals, amino acids, lipids).

Catechins represent a group of compounds which make up the hydrogenated flavones or anthocyanidines, and which represent derivatives of “catechins” (catechol, 3,3′,4′,5,7-flavanpentaol, 2-(3,4-dihydroxyphenyl)-chroman-3,5,7-triol). Epicatechine ((2R,3R)-3,3′,4′,5,7-Flavanpentaol) is another advantageous active substance within the meaning of the present invention.

Furthermore, polyphenoles or catechins from the group (−)-catechin, (+)-catechin, (−)-catechingallate, (−)-gallocatechingallate, (+)-epicatechin, (−)-epicatechin, (−)-epicatechin gallat, (−)-epigallocatechin, (−)-epigallocatechingallate are also advantageous active substances.

Flavone and its derivatives (often known collectively as “flavones”) are further advantageous active substances within the meaning of the present invention. They are characterised by the following basic structure (substitution provisions specified):

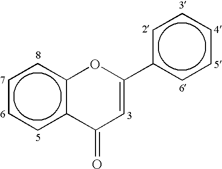

Some of the most important flavones, which can likewise be advantageously deployed in preparations within the meaning of the present invention, are listed in the following table:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kämpferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In the natural world, flavones generally occur in glycosylised form.

Within the framework of the present invention, the flavonoides are preferably chosen from the group of substances with the generic structural formula

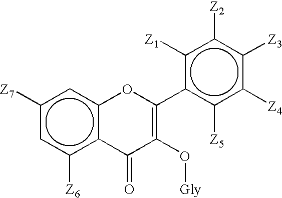

whereby Z₁ to Z₇ are selected independently of each other from the group H, OH, alkoxy- as well as hydroxyalkoxy-, whereby the alkoxy- or hydroxyalkoxy-groups can be branched and unbranched, and can have 1 to 18 C atoms, and whereby gly is chosen from the group of the monoglycoside and oligoglycoside residues.

Within the framework of the invention, however, flavonoides can also be advantageously selected from the group of substances with the general structural formula

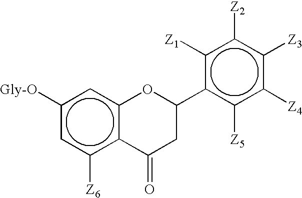

whereby Z₁ to Z₆ are selected independently of each other from the group H, OH, alkoxy- as well as hydroxyalkoxy-, whereby the alkoxy- or hydroxyalkoxy-groups can be branched or unbranched, and can have 1 to 18 C atoms, and whereby gly is selected from the group of monoglycoside and oligoglycoside residues.

Such structures can be advantageously selected from the group of substances with the generic structural formula

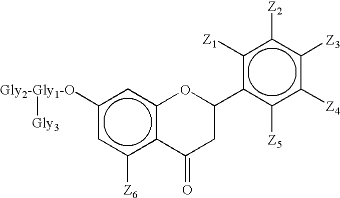

whereby gly₁, gly₂ and gly₃ independently represent monoglycoside residues. Gly₂ or gly₃ can also represent individual or joint saturations through hydrogen atoms.

Gly₁, gly₂ and gly₃ are advantageously selected independently of each other from the group of hexosyl residues, in particular the rhamnosyl residues and glucosyl residues. However, other hexosyl residues, for example allosyl, altrosyl, galactosyl, gulosyl, idosyl, mannosyl and talosyl can also be used advantageously. Within the framework of the present invention it can also be advantageous to use pentosyl residues.

It is advantageous to select Z₁ to Z₅ independently of each other from the group H, OH, methoxy-, ethoxy- as well as 2-hydroxyethoxy-, while the flavonglycosides have the structure

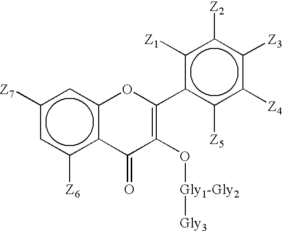

Within the framework of the present invention, it is particularly advantageous to select the flavonglycosides from the group that is represented by the following structure:

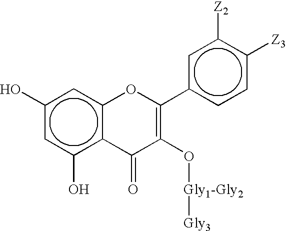

whereby gly₁, gly₂ and gly₃ independently represent monoglycoside residues. Gly₂ or gly₃ can also represent individual or joint saturations through hydrogen atoms.

Gly₁, gly₂ and gly₃ are advantageously selected independently of each other from the group of hexosyl residues, in particular the rhamnosyl residues and glucosyl residues. However, other hexosyl residues, for example allosyl, altrosyl, galactosyl, gulosyl, idosyl, mannosyl and talosyl can also be used advantageously. Within the framework of the present invention it can also be advantageous to use pentosyl residues.

Within the meaning of the present invention it is particularly advantageous to select the flavonglycoside(s) from the group of α-glucosylrutins, α-glucosylmyricetins, α-glucosylisoquercitrins, α-glucosylisoquercetins and α-glucosylquercitrins. Within the framework of the present invention, α-glucosylrutin is particularly advantageous.

Within the meaning of the present invention, naringin (aurantiin, naringenin-7-rhamnoglucoside), hesperidin (3′,5,7-trihydroxy-4′-methoxyflavanon-7-rutinoside, hesperidoside, hesperetin-7-O-rutinoside), rutin (3,3′,4′,5,7-pentahydroxyflyvon-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)-flavon-3-(6-O-(6-deoxy-α-L-mannopyranosyl)-β-D-glucopyranoside)), monoxerutin (3,3′,4′,5-tetrahydroxy-7-(2-hydroxyethoxy)-flavon-3-(6-O-(6-deoxy-α-L-mannopyranosyl)-β-D-glucopyranoside)), dihydrorobinetin (3,3′,4′,5′,7-pentahydroxyflavanon), taxifolin (3,3′,4′,5,7-pentahydroxyflavanon), eriodictyol-7-glucoside (3′,4′,5,7-tetrahydroxyflavanon-7-glucoside), flavanomareïn (3′,4′,7,8-tetrahydroxyflavanon-7-glucoside) and isoquercetin (3,3′,4′,5,7-pentahydroxyflavanon-3-(β-D-glucopyranoside) are also advantageous.

It is also advantageous to select the active substance(s) from the group of ubichinones and plastochinones.

Ubichinones are characterised by the structural formula

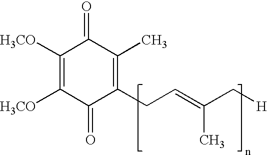

and represent the most widespread and consequently best studied biochinones. Depending upon the particular number of isoprene units linked in the side-chain, ubichinones are called Q-1, Q-2, Q-3 etc., or in accordance with the particular number of C atoms are known as U-5, U-10, U-15 etc. They appear preferably with specific chain lengths, e.g. in certain microorganisms and yeasts with n=6. In the case of the majority of mammals, including humans, Q10 predominates.

Particularly advantageous is coenzyme Q10, which is characterised by the following structural formula:

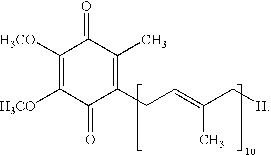

Plastochinones have the general structural formula

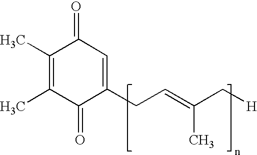

whereby they are distinguished by the number n of isoprene residues, and are named correspondingly, e.g. PQ-9 (n=9). Furthermore, other plastochinones with different substitutes on the chinon ring also exist.

Creatine and/or creatine derivatives are likewise favoured active substances within the meaning of the present invention. Creatine is characterised by the following structure:

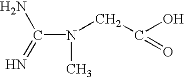

Favoured derivatives are creatine phosphate as well as creatine sulphate, creatine acetate, creatine ascorbate and the esterised derivatives of the carboxyl group with monofunctional or polyfunctional alcohols.

A further advantageous active substance is L-carnitine [3-hydroxy-4-(trimethylammonio)-isobutyric acid betaine]. Other advantageous active substances within the meaning of the present invention include the acyl-carnitines, which are selected from the group of substances with the following general structural formula

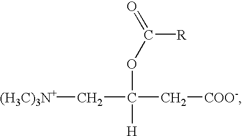

whereby R is selected from the group of branched and unbranched alkyl residues with up to 10 carbon atoms. Propionylcarnitin and in particular acetylcarnitin are favoured. Both entantiomeres (D and L form) can be used advantageously within the meaning of the present invention. It can also be advantageous to use any enantiomere mixture, for example a racemate comprising the D and L form.

Further advantageous active substances are sericoside, pyridoxol, aminoguadine, phytochelatine, isoflavones (genistein, daidzein, daidzin, glycitin), niacin, tyrosinsulphate, dioic acid, adenosin, pyridoxin, arginin, vitamin K, biotin and aromatic substances, sericosids as well as combinations of the aforementioned active substances.

In addition, one of the most important tasks of cosmetic and/or dermatological preparations is to moisturise and to regulate the moisture of the skin. For this purpose, in addition to water as a component of all emulsions, moisturisers are added to the preparations. Synthetic moisturisers are substitute substances for natural moisturising factor (NMF), which is made up of 40% free amino acids, 12% pyroglutamine acid, 12% lactates, 7% urea, 1.5% uric acid as well as flucosamin, creatinin and a variety of salts.

In addition to hydrolysed proteins, above all polyoles (multivalent alcohols) are used as synthetic moisturisers.

The most important representative of the polyoles is glycerine (glycerol, 1,2,3-propantriol), a colourless and odourless, sweet-tasting liquid. Glycerine has the following structure:

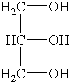

A further important representative of the polyoles is sorbit, a pentavelent alcohol which is found in rowan berries, and which can be obtained synthetically by reducing glucose. Sorbit is characterised by the following structure:

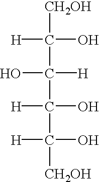

In particular, moisturisers containing chitosan, fucogel, lactic acid, propylenglycol, sorbitol, polyethylenglycol, dipropylenglycol, butylenglycol, mannitol, sodium pyrolidon carboxylic acid, glycin hyaluronic acid and their salts, amino acids such as glycin, urea, sodium and potassium salts can be bound to the skin more easily. Through the use of waxes (ester waxes, triglyceride waxes, ethoxlised waxes etc.) in the oil phase or as the oil phase, the adhesion of the active substances and moisturisers can be improved still further.

The moisture content of the skin can be determined using corneometric measurements. This entails using a corneometer in order to assess the dielectric characteristics of the stratum corneum. The corneometer consists of a scattering condenser whose capacity is (co-)determined by the dielectric characteristics of the stratum corneum. In order to determine how long a skin moisturisation brought about by a cosmetic and/or dermatological preparation remains efficacious, the moisture content of the skin is determined under constant measurement conditions, both prior to as well as two hours after the application of the cosmetic and/or dermatological preparation. These studies have demonstrated that the preparation containing moisturiser which is the subject of the present invention exerts a positive influence on skin moisture levels that is extremely pleasant for skin which has been damaged by skin irritation.

Of course, the list of the specified additional active substances or combinations of active substances that can be used in the preparations that are the subject of the present invention is not limiting. The active substances can be used either individually or in any desired combination with each other.

It may also be possible and advantageous to use the preparations that are the subject of the present invention as the basis for pharmaceutical formulations. Mutatis mutandis, corresponding requirements apply to the formulation of medical preparations. In this respect, the distinction between pure cosmetics and pure pharmaceuticals is flexible. Within the framework of the present invention, all classes of active substances are suitable as pharmaceutical active substances, whereby lipophilic active substances are favoured. Examples include: antihistamines, antiphlogistics, antibiotics, antimycotics, active substances which promote the flow of blood, ceratolytics, antihistamines, antiphlogistics, antibiotics, antimycotics, active substances which promote the flow of blood, ceratolytics, hormones, steroids, vitamins, hormones, steroids, vitamins etc.

It is also possible and advantageous to work repellents into the preparations that are the subject of the present invention. Particularly advantageous repellent active substances within the meaning of the present invention are the aforementioned active substances N,N-diethyl-3-methylbenzamide, 3-(N-n-butyl-N-acetyl-amino)propionic acid ethylester, 1-piperidine carboxylic acid 2-(2-hydroxyethyl)-1-methylpropylester and dimethylphthalate.

It is likewise advantageous within the meaning of the present invention to create cosmetic and dermatological preparations that also have the purpose of providing protection against the effects of sunlight. For this purpose, standard UV-A or UV-B filter substances are worked into the preparations. In addition, UV protective substances, as well as antioxidants and, if desired, preservatives, represent an effective protection of the preparations themselves against perishing.

The preparations within the meaning of the present invention accordingly contain preferably at least one UV-A, UV-B and/or broadband filter substance. The formulations may also, although this is not essential, contain one or several organic and/or inorganic pigments as UV filter substances, which can be present in the water and/or in the oil phase.

Within the meaning of the present invention, liquid UV filter substances which are liquid at room temperature are particularly advantageous, such as homomenthylsalicylate (INCI: homosalicylate), 2-ethylhexyl-2-cyano-3,3-diphenylacrylate (INCI: octocrylene), 2-ethylhexyl-2-hydroxybenzoate (2-ethylhexylsalicylate, ethylhexylsalicylate, INCI: ethylhexyl salicylate) and esters of cinnamic acid, preferably 4-methoxy cinnamic acid (2-ethylhexyl)ester (2-ethylhexyl-4-methoxycinnamate, INCI: ethylhexyl methoxycinnamate) and 4-methoxy cinnamic acid isopentylester (isopentyl-4-methoxycinnamate, INCI: isoamyl p-methoxycinnamate), 3-(4-(2,2-to ethoxycarbonylvinyl)-phenoxy)propenyl)-methoxysiloxan/dimethylsiloxan—copolymer which is available, for example, under the trade name Parsol® SLX from Hoffmann La Roche.

Favoured inorganic pigments are metal oxides and/or other metal compounds which are difficult to dissolve or which are insoluble in water, in particular the oxides of titanium (TiO₂), zinc (ZnO), iron (e.g. Fe₂O₃), zirconium (ZrO₂), silicium (SiO₂), manganese (e.g. MnO), aluminium (Al₂O₃), cerium (e.g. Ce₂O₃), mixed oxides of the corresponding metals as well as blends of such oxides as well as barium sulphate (BaSO₄).

Within the meaning of the present invention the pigments can also be used advantageously in the form of commercially available oily or aqueous preliminary dispersions. It can be advantageous for these preliminary dispersions to contain dispersal agents and/or solubilisation agents.

Within the framework of the present invention the pigments can be surface-treated (coated), whereby for example a hydrophilic, amphiphilic or hydrophobic character can be created or maintained. This surface treatment can entail using established processes to give the pigments a thin hydrophilic and/or hydrophobic inorganic and/or organic layer. Within the meaning of the present invention, the various surface coatings can also contain water.

Inorganic coatings within the meaning of the present invention can consist of aluminium oxide (Al₂O₃), aluminium hydroxide Al(OH)₃, or aluminium oxide hydrate (also: alumina, CAS-No. 1333-84-2), sodium hexametaphosphate (NaPO₃)₆, sodium metaphosphate (NaPO₃)ₙ, silicium dioxide (SiO₂) (also: silica, CAS-No. 7631-86-9), or iron oxide (Fe₂O₃). These inorganic coatings can be used singly, in combination and/or in combination with organic coating materials.

Organic coatings within the meaning of the present invention can consist of plant or animal aluminium stearate, plant or animal stearic acid, lauric acid, dimethylpolysiloxane (also: dimethicone), methylpolysiloxane (methicone), simethicone (a mixture of dimethylpolysiloxane with an average chain length of 200 to 350 dimethylsiloxane units and silica gel) or alginic acid. These organic coatings can be used singly, in combination and/or in combination with inorganic coating materials.

In accordance with the present invention, suitable tin oxide particles and preliminary dispersions of tin oxide particles are available under the following names from the companies listed below:

| Trade name | Coating | Manufacturer |
|---|---|---|
| Z-Cote HP1 | 2% Dimethicone | BASF |
| ZnO NDM | 5% Dimethicone | H&R |
| MZ-303S | 3% Methicone | Tayca Corporation |
| MZ-505S | 5% Methicone | Tayca Corporation |

Suitable titanium oxide particles and preliminary dispersions of titanium oxide particles are available under the following names from the companies listed below:

| Trade name | Coating | Manufacturer |
|---|---|---|
| MT-100TV | Aluminium hydroxide/stearic | Tayca Corporation |
| | acid | |
| MT-100Z | Aluminium hydroxide/stearic | Tayca Corporation |
| | acid | |
| Eusolex T-2000 | Alumina/simethicone | Merck KgaA |
| Titandioxid T805 | Octyltrimethylsilane | Degussa |
| (Uvinul TiO₂) | | |
| MT-100AQ | Silica/aluminium hydroxide/ | Tayca Corporation |
| | alginic acid | |
| Eusolex T-Aqua | Water/alumina/sodium | Merck KgaA |
| | metaphosphate | |

Advantageous UV-A filter substances within the meaning of the present invention are dibenzoylmethane derivatives, in particular 4-(tert.-butyl)-4′-methoxydibenzoylmethane (CAS-No. 70356-09-1), which is sold by Givaudan under the brand name Parsol® 1789 and by Merck under the trade name Eusolex® 9020.

Further advantageous UV filter substances within the meaning of the present invention are sulphonated, water-soluble UV filters, such as e.g.:

Phenylen-1,4-bis-(2-benzimidazyl)-3,3′-5,5′-tetra sulphonic acid and its salts, particularly the corresponding sodium, potassium or triethanolammonium salts, in particular the phenylen-1,4-bis-(2-benzimideazyl)-3,3′-5,5′-tetra sulphonic acid to sodium salt with the INCI name bisimidazylate (CAS-No. 180898-37-7), which is available for example under the trade name Neo Heliopan AP from Haarmann & Reimer;

Salts of 2-phenylbenzimidazol-5-sulphonic acid, such as their sodium, potassium or triethanolammonium salts, as well as sulphonic acid itself with the INCI name phenylbenzimidazole sulphonic acid (CAS.-No. 27503-81-7), which is available for example under the trade name Eusolex 232 from Merck or under the trade name Neo Heliopan Hydro from Haarmann & Reimer;

1,4-di(2-oxo-10-sulpho-3-bornylidenmethyl)-benzol (also: 3,3′-(1,4-phenylendimethylene)-bis-(7,7-dimethyl-2-oxo-bicyclo-[2.2.1]hept-1-ylmethane sulphonic acid) and its salts (in particular the corresponding 10-sulphato compounds, in particular the corresponding sodium, potassium or triethanolammonium salt), which is also known as benzol-1,4-di(2-oxo-3-bornylidenmethyl-10-sulphonic acid). Benzol-1,4-di(2-oxo-3-bornylidenmethyl-10-sulphonic acid) has the INCI name terephtalidene dicampher sulphonic acid (CAS.-No. 90457-82-2) and is available for example under the trade name Mexoryl SX from the company Chimex;

Sulphonic acid derivatives of 3-benzylidencampher, such as e.g. 4-(2-oxo-3-bornylidenmethyl)benzol sulphonic acid, 2-methyl-5-(2-oxo-3-bornylidenmethyl) sulphonic acid and their salts.

Advantageous UV filter substances within the meaning of the present invention are furthermore so-called broadband filters, that is to say, filter substances which absorb both UV-A as well as UV-B radiation.

Advantageous broadband filters or UV-B filter substances are for example triazine derivatives, such as e.g.:

2,4-bis-{[4-(2-ethyl-hexyloxy)-2-hydroxy]-phenyl}-6-(4-methoxyphenyl)-1,3,5-triazine (INCI: bis-ethylhexyloxyphenol methoxyphenyl triazine), which is available under the trade name Tinosorb® S from the company CIBA-Chemikalien GmbH;

Diethylhexylbutylamidotriazone (INCI: diethylhexyl butamido triazone), which is available under the trade name UVASORB HEB from Sigma 3V;

4,4′,4″-(1,3,5-triazine-2,4,6-triyltriimino)-tris-benzoic acid tris(2-ethylhexylester), also known as: 2,4,6-tris-[anilino-(p-carbo-2′-ethyl-1′-hexyloxy)]-1,3,5-triazine (INCI: ethylhexyl triazone), which is distributed by the company BASF Aktiengesellschaft under the trade name UVINUL® T 150.

Further advantageous triazine derivatives within the meaning of the present invention are 2,4-bis-{[4-(3-sulphonato)-2-hydroxy-propyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethyl-hexyloxy)-2-hydroxy]-phenyl}-6-[4-(2-methoxyethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]-phenyl}-6-(4-methoxyphenyl)-1,3,5-triazine and 2,4-bis-{[4-(1′,1′, 1′,3′,5′,5′,5′-heptamethylsiloxy-2″-methyl-propyloxy)-2-hydroxy]-phenyl}-6-(4-methoxyphenyl)-1,3,5-triazine.

An advantageous benzotriazol within the meaning of the present invention is 2,2′-methylene-bis-(6-(2H-benzotriazol-2-yl)-4-(1,1,3,3-tetramethylbutyl)-phenol), a broadband filter which is characterised by the chemical structural formula

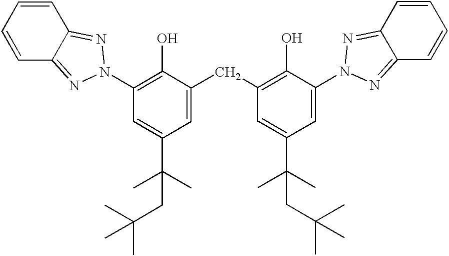

and which is available under the trade name Tinosorb® M from the company CIBA-Chemikalien GmbH.

An advantageous benzotriazol within the meaning of the present invention is furthermore 2-(2H-benzotriazol-2-yl)-4-methyl-6-[2-methyl-3-[1,3,3,3-tetramethyl-1-[(trimethylsilyl)oxy]disiloxanyl]-propyl]-phenol (CAS-No. 155633-54-8) with the INCI name of drometrizole trisiloxane, which is characterised by the chemical structural formula.

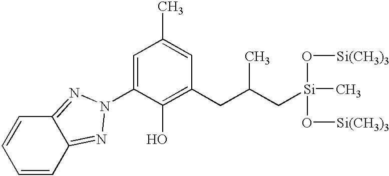

Further advantageous benzotriazols within the meaning of the present invention are [2,4′-dihydroxy-3-(2H-benzotriazol-2-yl)-5-(1,1,3,3-tetramethylbutyl)-2′-n-octoxy-5′-benzoyl]diphenylmethane, 2,2′methylen-bis-[6-(2H-benzotriazol-2-yl)-4-(methyl)phenol], 2,2′-methylene-bis-[6-(2H-benzotiazol-2-yl)-4-(1,1,3,3-trtramethylbutyl)phenol], 2-(2′-hydroxy-5′-octylphenyl)-benzotriazol, 2-(2′-hydroxy-3′,5′-di-t-amylphenyl)benzotriazol and 2-(2′-hydroxy-5′-methylphenyl)benzotriazol.

The further UV filter substances can be oil-soluble or water-soluble.

Advantageous oil-soluble UV-B and/or broadband filter substances within the meaning of the present invention are e.g.:

3-benzylidencampher derivatives, preferably 3-(4-methylbenzyliden)camphor, 3-benzylidencamphor;

4-amino benzoic acid derivatives, preferably 4-(dimethylamino) benzoic acid (2-ethylhexyl)ester, 4-(dimethylamino) benzoic acid amylester;

3-(4-(2,2-bis ethoxycarbonylvinyl)-phenoxy)propenyl)-methoxysiloxan/dimethylsiloxan—co-polymer which is available for example under the trade name Parsol® SLX from Hoffmann La Roche.

A further light protection filter substance which can be used advantageously within the framework of the present invention is ethylhexyl-2-cyano-3,3-diphenylacrylate (octocrylen), which is available from BASF under the name Uvinul® N 539.

Hydroxybenzophenone, in particular aminobenzophenone, in combination with acrylamide polymers or co-polymers also represent suitable light protection filters, and can consequently be contained advantageously in the preparations which are the subject of the present invention.

Of course, the list of specified UV filters that can be used within the meaning of the present invention is not intended to be limiting.

It is advantageous for the preparations which are the subject of the present invention to contain the substances which absorb UV radiation in the UV-A and/or UV-B range in a total quantity of e.g. 0.1 w/w % to 30 w/w %, preferably 0.5 to 20 w/w %, in particular 1.0 to 15.0 w/w %, in each case with respect to the total weight of the preparations, in order to make cosmetic preparations available which protect the skin from the entire range of ultraviolet light.

The cosmetic and dermatological preparations in accordance with the present invention can contain cosmetic agents of the sort which are customarily used in such preparations, e.g. preservatives, preservative agents, bactericides, perfumes, antifoaming agents, thickening agents and/or fillers which improve skin sensation.

The preparation which is the subject of the present invention can applied in the form of a gel, a cream, a lotion, a spray, a drug delivery system or via saturated cloths.

In a technical sense, gels mean: easily malleable dispersal systems of relatively stable form comprising at least two components, which are generally composed of a—mostly solid—colloidally-divided substance made of long-chained molecular groupings (e.g. gelatine, silic acid, polysaccharides) to form the lattice, and a liquid dispersion agent (e.g. water). The colloidally-divided substance is often called a thickening or gelling agent. It forms a spatial network within the dispersion agent, whereby individual colloidally present particles can be linked together in varying degrees of strength by means of electrostatic interaction. The dispersion agent which surrounds the network is characterised by an electrostatic affinity to the gelling agent, that is to say, a primarily polar (in particular: hydrophilic) gelling agent preferably gels a polar dispersion agent (in particular water), while a primarily non-polar gelling agent preferably gels a non-polar dispersion agent.

Powerful electrostatic interactions which are realised for example in hydrogen-bridged compounds between the gelling agent and the dispersion agent, as well as between the dispersion agent molecules themselves, can give the dispersion agent strong structures. Hydrogels can consist of almost 100% water (in addition to, for example, approx. 0.2-1.0% of gelling agent), and yet can still have a solid consistency. In such cases, the water component forms ice-like structural elements, which means that the etymological origin of the name gel [from the Latin “gelatum”=“frozen”, through the alchemistic term “gelatina” (16^{th} century) to the modern “gelatine”] is certainly justified.

In addition, in the cosmetic and pharmaceutical fields, lipogels and oleogels (made of waxes, fats and fatty oils) as well as carbogels (made of paraffin or petrolatum) are common. In practical terms, a distinction is drawn between oleogels, which are practically water-free, and hydrogels, which are practically fat-free. Gels tend to be transparent. In the cosmetic and pharmaceutical fields, gels tend to have a semi-solid, often flowable consistency.

Furthermore, so-called tenside gels are common preparations within the state-of-the-art. These are systems, which in addition to water also contain a high concentration of emulsifiers, typically more than approx. 25 w/w %, in relation to the total composition. If oil components are dissolved in these tenside gels, which are also known as “surfactant gels”, then this results in micro emulsion gels that are also called “ringing gels”. Through the addition of non-ionic emulsifiers, for example alkylpolyglycosides, cosmetically more elegant microemulsion gels can be created.

It is known that by linking together the droplets of a low-viscosity, in particular highly liquid microemulsion with latticed substances, the three-dimensional network of a gel can be produced.

Creams are understood to mean non-free-flowing preparations which have a viscosity, at 25° C., of more than 10,000 mPa*s (viscosity tester VT-O₂, from the company Haake). Lotions are understood to mean free-flowing preparations which have a viscosity, at 25° C., of 2,000-10,000 mPa*s (viscosity tester VT-O₂, from the company Haake).

Furthermore, the preparations that are the subject of the present invention are also ideal for use as saturation media for cloths and fabrics that can be used by consumers either dry or wet. In this respect, the compositions with the ultrafine droplets attach easily to the fibres, which is viewed as an advantage.

It is also possible to make the preparations that are the subject of the present invention available in the form of drug delivery systems. Drug delivery systems are plaster-like forms of drugs that contain the active substance in the form of a reservoir from which the active substance can be released over a longer period of time. Because of these characteristics, and irrespective of the concentration of the active substance in the reservoir, drug delivery systems are able to supply the organism with a constant quantity of active substances per time unit over a lengthy period of time. For this reason, they are classified as retard drugs.

When it comes to drug delivery systems, a distinction is made between topical and transdermal delivery. The topical formulations contain active substances whose release and effect is limited to the area directly below and in the vicinity of the application site. Transdermal formulations, by contrast, contain active substances which are applied through the skin, and which penetrate the vascular system to reach the entire organism in order to generate an effective expression of the active substance.

Relative to other drug forms (e.g. creams, sprays, suppositories, tablets), drug delivery systems offer a variety of advantages:

Because drug delivery systems remain effective for long periods of time, patient compliance is significantly improved relative to drug forms that need to be applied several times during the course of the day.

The application of the drug delivery systems can make it possible to reduce the dose, which can result in a reduction in side effects caused by active substances that e.g. have limited therapeutic range.

In the event of side effects appearing, the application of the drug can be stopped immediately by simply removing the drug delivery system.

The continuous release of the active substance prevents swings in active substance concentrations of the sort that occur in the area of the skin and in the serum when such substances are applied at set intervals during the day.

In comparison to oral drug forms, transdermal application reduces the first-pass metabolism of the drug substance, as the metabolism in the area of the skin is significantly lower in comparison to the metabolism in the stomach and liver. As a consequence, drug doses given using drug delivery systems are lower than in the case of oral drug forms.

In the case of drug delivery systems, in contrast to oral drugs, no account needs to be taken of the influence of food on the penetration of the drug.

The dosage can easily be defined by means of the surface area of the drug delivery system.

The manufacture of the preparations which are the subject of the present invention is performed advantageously by bringing a mixture of the basic components, comprising the water phase, oil phase, one or more of the O/W emulsifiers within the framework of the present invention, if desired one or more co-emulsifiers, as well as if desired further agents, additives and/or active substances, to a temperature which corresponds at least to the melting temperature of the oil component with the highest melting point, and then by cooling the formed emulsion back to room temperature, whereby in accordance with the framework of the present invention the active substances polidocanol and essential oils and possible thickeners can be added at any time during the manufacturing process. Preferably, the entire procedure is performed while stirring.

The following examples are designed to illustrate the present invention, without however restricting it. All specified quantities, concentrations and percentage ratios, insofar as not otherwise stated, relate to the weight and the total quantity or to the total weight of the preparations.

### EXAMPLES

O/W emulsions

| | 1 | 2 | 3 | 4 | 5 |
|---|---|---|---|---|---|
| Cetylalcohol | 1.0 | 0.5 | 1.0 | 2.0 | 3.0 |
| Glycerylstearate | 1.0 | 1.5 | 1.0 | 3.0 | 2.0 |
| Paraffin oil | 2.5 | 1.0 | 4.0 | 2.5 | 1.5 |
| Decyloleate | 0.5 | 0.75 | — | 2.0 | 0.25 |
| Octyldodecanol | — | 1.0 | — | 3.0 | 0.25 |
| Dicaprylylcarbonate | 0.1 | 0.05 | — | 0.5 | — |
| Glycerine | 3.0 | 5.0 | 12.0 | 15.0 | 1.5 |
| Dimethicone | 0.6 | 0.3 | 1.0 | 1.2 | 1.8 |
| Perfume | q,s, | q,s, | q,s, | q,s, | q,s, |
| Evening primrose oil | 2.0 | — | 1.0 | — | 8.0 |
| Caprylic/caprinic acid triglyceride | 2.0 | 2.5 | 6.0 | 5.0 | 0.5 |
| Methylparabene | 0.1 | 0.15 | — | 0.03 | 0.14 |
| Polydocanol | 1.0 | 1.0 | 2.0 | 2.5 | — |
| Menthol | 0.05 | — | 0.15 | 0.25 | 0.3 |
| Polyethylenglycol(12)cetearylether | 1.0 | 2.2 | 2.6 | 2.4 | — |
| Cyclomethicone | 0.5 | 0.75 | 1.0 | 2.0 | 0.25 |
| Ethanol | 1 | 5 | — | 3 | 2 |
| Modified starch | — | 2.5 | — | 0.15 | — |
| Propylparabene | 0.1 | 0.15 | — | 0.15 | — |
| Iodopropynylbutylcarbamate + | — | — | 0.3 | — | 0.1 |
| DMDM hydantoin | | | | | |
| Water | ad 100 | ad 100 | ad 100 | ad 100 | ad 100 |

O/W emulsions

| | 6 | 7 | 8 | 9 | 10 |
|---|---|---|---|---|---|
| PEG-40-stearate | — | 0.5 | — | — | 1.0 |
| Panthenol | — | 0.75 | 1.0 | 2.0 | 0.25 |
| Octyldodecanol | — | 1.0 | 0.75 | 3.0 | 0.25 |
| Dicaprylylcarbonate | — | 0.05 | 0.15 | 0.5 | 1.0 |
| Glycerine | 3.0 | 5.0 | 10.0 | 15.0 | 1.5 |
| Lactic acid | 0.6 | 0.3 | 0.15 | 1.2 | 1.8 |
| Perfume | q,s, | q,s, | q,s, | q,s, | q,s, |
| Polydocanol | 3.0 | 1.0 | 0.5 | 2.5 | 5.0 |
| Menthol | 0.1 | 1.0 | 0.5 | 0.25 | 0.5 |
| Cetylalcohol | 1.0 | 0.5 | 1.0 | 2.0 | 3.0 |
| Glycerylstearate | 1.0 | 1.5 | 1.0 | 3.0 | 2.0 |
| Potassium sorbate | — | — | 0.3 | — | 0.15 |
| Caprylic/caprinic acid triglyceride | — | 2.5 | 3.0 | 5.0 | 0.5 |
| Methylparabene | — | 0.15 | 0.05 | 0.3 | 0.4 |
| Propylparabene | — | 0.4 | 0.25 | 0.15 | — |
| Water | ad 100 | ad 100 | ad 100 | ad 100 | ad 100 |

O/W emulsions

| | 11 | 12 | 13 | 14 | 15 |
|---|---|---|---|---|---|
| Cetylalcohol | 1.0 | 0.5 | 1.0 | 2.0 | 3.0 |
| Glycerylstearate | 1.0 | 1.5 | 1.0 | 3.0 | 2.0 |
| Polydocanol | 1.0 | 1.0 | 1.5 | 0.5 | 0.75 |
| Menthol | 0.5 | 0.1 | 0.05 | 0.25 | 0.2 |
| Paraffin oil | 2.5 | — | 8.0 | 2.5 | 1.5 |
| Cocoglyceride | 8.0 | 6.0 | 0.5 | — | 2.5 |
| PEG-40 hydrated castor oil | 1.0 | 1.5 | — | 1.8 | — |
| Hydrated polyisobutene | — | 0.75 | 1.0 | 2.0 | 0.25 |
| Octyldodecanol | 0.5 | 1.0 | 0.75 | 8.0 | 0.25 |
| Dicaprylylcarbonate | 0.1 | 0.05 | 0.15 | 0.5 | 1.0 |
| Glycerine | 3.0 | 5.0 | — | 15.0 | 10.0 |
| Perfume | q,s, | q,s, | q,s, | q,s, | q,s, |
| Butylenglykol | 5.0 | — | 15.0 | — | 8.0 |
| Caprylic/caprinic acid triglyceride | 2.0 | 2.5 | 3.0 | 5.0 | 0.5 |
| Methylparabene | 0.4 | — | 0.05 | 0.3 | 0.4 |
| Propylparabene | 0.3 | 0.4 | 0.25 | 0.15 | — |
| Water | ad 100 | ad 100 | ad 100 | ad 100 | ad 100 |

O/W emulsions

| | 16 | 17 | 18 | 19 | 20 |
|---|---|---|---|---|---|
| Polyethylenglycol(12)cetearylether | 1.0 | 0.5 | — | — | — |
| Cyclomethicone | 0.5 | 0.75 | 1.0 | 2.0 | 0.25 |
| Octyldodecanol | 0.5 | 1.0 | 0.75 | 3.0 | 0.25 |
| Dicaprylylcarbonate | 0.1 | 1.0 | 15.0 | 0.5 | 1.0 |
| Dimethicone | — | — | — | 5.0 | 1.0 |
| Glycerine | 3.0 | 5.0 | 10.0 | 15.0 | 1.5 |
| Polydocanol | 1.0 | 1.0 | 5 | 2.5 | 10 |
| Lanolinalcohol | 0.25 | 0.5 | 1.5 | 3.0 | 1.0 |
| Menthol | 0.05 | 0.5 | 0.25 | 0.3 | 0.5 |
| Paraffin oil | 2.5 | 1.0 | 8.0 | 2.5 | 1.5 |
| Perfume | q,s, | q,s, | q,s, | q,s, | q,s, |
| Tocopherol | 1.0 | 0.5 | 1.5 | 3.0 | 0.5 |
| Caprylic/caprinic acid triglyceride | 2.0 | 2.5 | 3.0 | 5.0 | 0.5 |
| Methylparabene | 0.4 | 0.15 | 0.05 | 0.3 | 0.4 |
| Propylparabene | 0.3 | 0.4 | 0.25 | 0.15 | — |
| Modified starch | — | 2.5 | — | 0.15 | 5.0 |
| Water | ad 100 | ad 100 | ad 100 | ad 100 | ad 100 |

When applied to irritated skin, all the listed examples of preparations have the immediate effect of reducing or eliminating itching. Furthermore, the preparations that are the subject of the present invention can be applied to the skin with ease, and are pleasant to spread.

1. A cosmetic or dermatological preparation comprising:
water,
one or more O/W emulsifiers,
optionally one or more co-emulsifiers,
one or more oil phases,
polidocanol and
an essential oil.

2. A cosmetic or dermatological preparation in accordance with claim 1, comprising monostearate polyoxyethylene (40) as an O/W emulsifier.

3. A cosmetic or dermatological preparation in accordance with claim 1, comprising glyceryl stearate as a co-emulsifier.

4. A cosmetic or dermatological preparation in accordance with claim 1, wherein the O/W emulsifiers or co-emulsifiers are selected from the group consisting of ethoxylised fatty alcohols, ethoxylised stearates and glycerine esters.

5. A cosmetic or dermatological preparation in accordance with claim 1, comprising menthol as an essential oil.

6. A cosmetic or dermatological preparation in accordance with claim 1, wherein polidocanol is used in a ratio of 0.01 to 20 w/w %.

7. A cosmetic or dermatological preparation in accordance with claim 6, wherein polidocanol is used in a ratio of 1 to 10 w/w %.

8. A cosmetic or dermatological preparation in accordance with claim 1, wherein the essential oils are used in a ratio of 0.001 to 10 w/w %.

9. A cosmetic or dermatological preparation in accordance with claim 8, wherein the essential oils are used in a ratio of 0.01 to 1 w/w %.

10. A cosmetic or dermatological preparation in accordance with claim 1, wherein the oil phase content is 1 to 50 w/w % in relation to the mass of the overall preparation.

11. A cosmetic or dermatological preparation in accordance with claim 10, wherein the oil phase content is 1 to 40 w/w % in relation to the mass of the overall preparation.

12. A cosmetic or dermatological preparation in accordance with claim 1, further comprising ribwort tincture.

13. A cosmetic or dermatological preparation in accordance with claim 1, further comprising a moisturiser.

14. A cosmetic or dermatological preparation in accordance with claim 13, wherein the moisturiser includes one or more polyols selected from the group consisting of glycerine, sorbite, urea and butylene glycol.

15. A cosmetic or dermatological preparation in accordance with claim 1, further comprising one or more cosmetic or dermatological agents selected from the group consisting of colourings, pigments, powders, hydrophilic and lipophilic active substances, deodorants, and light protection agents.

16. A cosmetic or dermatological preparation in accordance with claim 1, further comprising active substances selected from the group consisting of antioxidants, NO synthesis blockers, catechines, bile acid esters, flavones, flavonoids, ubichinones, plastochinones, creatines, creatine derivatives and repellents.

17. A cosmetic or dermatological preparation in accordance with claim 16, wherein the active substances include coenzyme Q10.

18. A cosmetic or dermatological preparation in accordance with claim 1, further comprising light protection filter substances that absorb radiation selected from the group consisting of UV-A and UV-B radiation.

19. A cosmetic or dermatological preparation in accordance with claim 1 in a form selected from the group consisting of an antipruritic cream, lotion, gel, spray and foam.

20. A drug delivery system containing a cosmetic or dermatological preparation in accordance with claim 1.

21. A saturated cloth containing a cosmetic or dermatological preparation in accordance with claim 1.

22. A method of treating a pruritic condition comprising applying a cosmetic or dermatological preparation in accordance with claim 1.

23. A cosmetic or dermatological preparation that includes a non-ionic emulsion comprising polyethyleneglycol(12)cetearylether, polidocanol and menthol.
